(19) United States
(12) Patent Application Publication
(10) Pub. No.: US 2021/0133653 A1
(43) Pub. Date: May 6, 2021

(54) INFORMATION PROCESSING APPARATUS AND NON-TRANSITORY COMPUTER READABLE MEDIUM

(71) Applicant: FUJI XEROX CO., LTD., Tokyo (JP)

(72) Inventors: Kengo TOKUCHI, Kanagawa (JP); Masahiro SATO, Kanagawa (JP); Hanako KARIYA, Kanagawa (JP)

(73) Assignee: FUJI XEROX CO., LTD., Tokyo (JP)

(21) Appl. No.: 16/838,606

(22) Filed: Apr. 2, 2020

(30) Foreign Application Priority Data
Oct. 30, 2019 (JP) 2019-197911

(51) Int. Cl. G06Q 10/06; G16H 40/67; G16H 50/30; A61B 5/0205; A61B 5/0476; A61B 5/0488; A61B 5/145

(52) U.S. Cl. CPC G06Q 10/06311; G16H 40/67; G16H 50/30; A61B 5/02108; A61B 5/0476; A61B 5/0488; A61B 5/14507; A61B 5/0205

(57) ABSTRACT

An information processing apparatus includes a processor configured to: obtain biological information of a person who has received a request to implement an activity, the biological information being generated from the person on receipt of the request to implement the activity; and estimate, on the basis of the biological information, whether the person is able to accept implementation of the activity.

### CROSS-REFERENCE TO RELATED APPLICATIONS

This application is based on and claims priority under 35 USC 119 from Japanese Patent Application No. 2019-197911 filed Oct. 30, 2019.

### BACKGROUND

### (i) Technical Field

The present disclosure relates to an information processing apparatus and a non-transitory computer readable medium.

### (ii) Related Art

Japanese Unexamined Patent Application Publication No. 2017-173899 describes a production management system including: a cloud unit that accumulates a person's health examination information; a sensor that detects a worker's physical condition data; and a controller that estimates the worker's physical condition on the basis of the physical condition data obtained from the sensor and the health examination information obtained from the cloud unit, and that adjusts a production plan on the basis of the estimation result.

### SUMMARY

Aspects of non-limiting embodiments of the present disclosure relate to estimating whether a person who has received a request to implement an activity is able to accept the implementation.

Aspects of certain non-limiting embodiments of the present disclosure address the above advantages and/or other advantages not described above. However, aspects of the non-limiting embodiments are not required to address the advantages described above, and aspects of the non-limiting embodiments of the present disclosure may not address advantages described above.

According to an aspect of the present disclosure, there is provided an information processing apparatus including a processor configured to: obtain biological information of a person who has received a request to implement an activity, the biological information being generated from the person on receipt of the request to implement the activity; and estimate, on the basis of the biological information, whether the person is able to accept implementation of the activity.

### BRIEF DESCRIPTION OF THE DRAWINGS

An exemplary embodiment of the present disclosure will be described in detail based on the following figures, wherein:

FIG. 1 is a block diagram illustrating the configuration of an information processing system according to an exemplary embodiment;

FIG. 2 is a block diagram illustrating the configuration of an information processing apparatus according to the present exemplary embodiment;

FIG. 3 is a flowchart illustrating a process performed by the information processing apparatus according to the present exemplary embodiment;

FIG. 4 is a diagram illustrating a biological response management table;

FIG. 5 is a diagram illustrating a history management table; and

FIG. 6 is a diagram illustrating a schedule management table.

### DETAILED DESCRIPTION

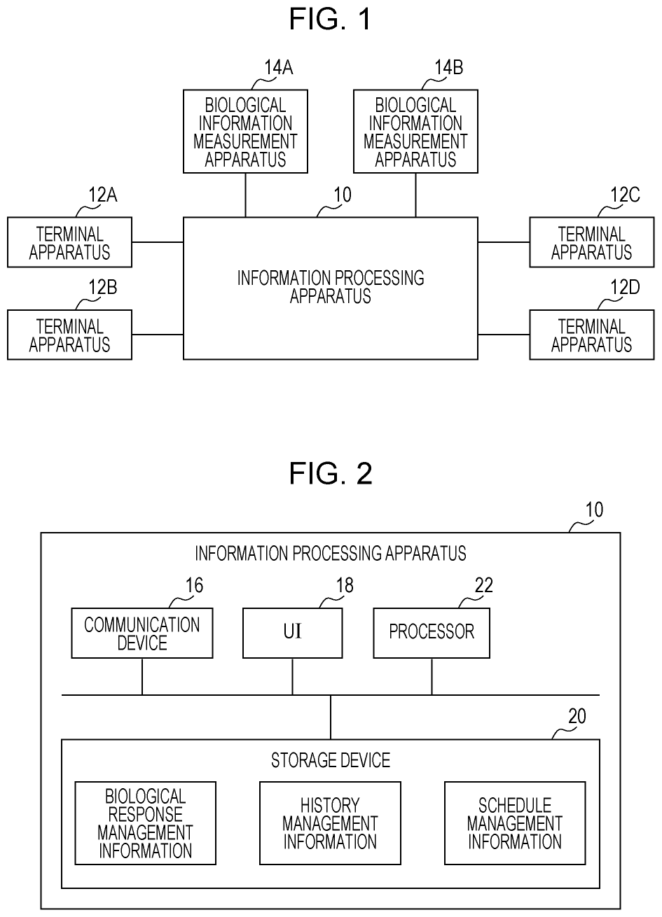

Referring to FIG. 1, an information processing system according to an exemplary embodiment will be described. FIG. 1 illustrates an example of the configuration of the information processing system according to the present exemplary embodiment.

The information processing system according to the present exemplary embodiment includes an information processing apparatus 10, one or more terminal apparatuses, and one or more biological information measurement apparatuses. In the example illustrated in FIG. 1, the information processing system includes terminal apparatuses 12A, 12B, 12C, and 12D, and biological information measurement apparatuses 14A and 14B; however, this is only one example. Hereinafter, the terminal apparatuses 12A, 12B, 12C, and 12D will be referred to as a “terminal apparatus 12” when it is not necessary to distinguish between the terminal apparatuses 12A, 12B, 12C, and 12D. In addition, the biological information measurement apparatuses 14A and 14B will be referred to as a “biological information measurement apparatus 14” when it is not necessary to distinguish between the biological information measurement apparatuses 14A and 14B. Note that the number of each type of apparatus illustrated in FIG. 1 is only one example, and the number of each type of apparatus is not limited to the number of each type of apparatus illustrated in FIG. 1. In addition, the information processing system may include another apparatus (for example, an external apparatus such as a server) other than those apparatuses illustrated in FIG. 1.

The information processing apparatus 10, the terminal apparatus 12, and the biological information measurement apparatus 14 are configured to communicate with another apparatus or the like. This communication may be wired communication using a cable, or wireless communication. In short, each apparatus may be physically connected to another apparatus or the like by a cable to transmit and receive information to and from each other, or to transmit and receive information to and from each other through wireless communication. As the wireless communication, short-range wireless communication, Wi-Fi (registered trademark), or the like is used. Alternatively, wireless communication based on standards other than the above may be used. Examples of short-range wireless communication include Bluetooth (registered trademark), radio frequency identifier (RFID), and near-field communication (NFC). Each apparatus may communicate with another apparatus or another sensor via a communication channel such as a local area network (LAN) or the Internet.

The information processing apparatus 10 is, for example, a personal computer (hereinafter abbreviated as “PC”), a tablet PC, a smartphone, a cellular phone, or another apparatus. The information processing apparatus 10 may be a terminal apparatus that may be carried by a user (such as a tablet PC, a smartphone, or a cellular phone), or an apparatus installed and used on a table or the like. The information processing apparatus 10 may be a smart speaker that has a communication function, a microphone, and a speaker. The information processing apparatus 10 may be installed indoors (such as on the floor or ceiling of a room, on a table, or the like) or may be installed outdoors. In addition, the information processing apparatus 10 may be a movable apparatus (such as a self-propelled apparatus) or a server.

The terminal apparatus 12 is, for example, a PC, a tablet PC, a smartphone, a cellular phone, or another apparatus. The terminal apparatus 12 may be a terminal apparatus that may be carried by a user (such as a tablet PC, a smartphone, or a cellular phone), or an apparatus installed and used on a table or the like.

The biological information measurement apparatus 14 has a sensor, an electrode, and the like, and is configured to measure a user's biological information. The individual biological information measurement apparatuses 14 may measure different types of biological information. Needless to say, one, some, or all of the biological information measurement apparatuses 14 may be configured to measure the same type of biological information. Alternatively, each biological information measurement apparatus 14 may be configured to measure one type of biological information, or may be configured to measure a plurality of types of biological information.

The biological information measurement apparatus 14 may transmit biological information measured by itself to the information processing apparatus 10 and/or the terminal apparatus 12. The biological information measurement apparatus 14 may transmit biological information to the information processing apparatus 10 and/or the terminal apparatus 12 whenever the biological information measurement apparatus 14 measures biological information, may store biological information and transmit the biological information to the information processing apparatus 10 and/or the terminal apparatus 12 at predetermined time intervals, or may transmit biological information to the information processing apparatus 10 and/or the terminal apparatus 12 at a time point specified by a user. In addition, the biological information measurement apparatus 14 may receive biological information measured by another biological information measurement apparatus 14 from this other biological information measurement apparatus 14, and may transmit biological information measured itself and the biological information measured by the other biological information measurement apparatus 14 to the information processing apparatus 10 and/or the terminal apparatus 12.

The biological information measurement apparatus 14 may analyze biological information measured by itself or by another biological information measurement apparatus 14, and transmit information indicating the analysis result to the information processing apparatus 10 and/or the terminal apparatus 12. For example, the biological information measurement apparatus 14 includes a processor, and the processor may analyze biological information. Needless to say, the analysis may be conducted by the information processing apparatus 10 or the terminal apparatus 12.

The biological information measurement apparatus 14 may include a battery and be driven by power supplied from the battery, or may receive power supplied from the information processing apparatus 10 or the terminal apparatus 12 and be driven by the received power.

The biological information measurement apparatus 14 may be a wearable apparatus that is entirely worn by a user and that measures biological information. For example, the biological information measurement apparatus 14 may be an apparatus worn on a user's head, a hearable apparatus worn on a user's ear, an apparatus worn on a user's arm, hand, wrist, or finger (such as a watch-type apparatus), an apparatus hung on a user's neck, or an apparatus worn on a user's body or leg.

Biological information includes various types of physiological information and anatomical information generated from a user who is a living body. The category of the concept of biological information includes, for example, information indicating brain activities (such as brain waves, brain blood flow, and brain magnetic field signals), information indicating a pulse rate, myoelectric information such as a myoelectric waveform, information regarding saliva, information indicating a pulse wave, information indicating a blood pressure, information indicating a pulse, information indicating a heart rate, information indicating an electrocardiographic waveform, information indicating eye movement, information indicating a body temperature, information indicating a sweat rate, information indicating the line of sight, audio information, and information indicating a user's movement. In addition, information identified by a biomarker may be used as biological information. These are only examples of biological information, and other physiological information and anatomical information may be used as biological information. The biological information measurement apparatus 14 may measure one type or a plurality of types of these items of biological information.

The information processing apparatus 10 receives biological information from the biological information measurement apparatus 14, analyzes the biological information, stores the biological information, outputs the biological information, stores information indicating the result of analysis of the biological information, and outputs information indicating the result of analysis of the biological information. Outputting biological information includes, for example, displaying the biological information, outputting the biological information as audio information, and transmitting the biological information to another apparatus. Outputting information indicating the result of analysis of biological information includes, for example, displaying information indicating the analysis result, outputting the analysis result as audio information, and transmitting information indicating the analysis result to another apparatus.

Analysis of biological information, storage of biological information, output of biological information, storage of information indicating the result of analysis of biological information, and output of information indicating the result of analysis of biological information may be performed by the terminal apparatus 12. Needless to say, analysis of biological information may be conducted by the biological information measurement apparatus 14.

The information processing apparatus 10 may include one or more biological information measurement apparatuses 14. In short, one or more biological information measurement apparatuses 14 may be incorporated in the information processing apparatus 10 to constitute one apparatus. The entire information processing apparatus 10 including one or more biological information measurement apparatuses 14 may be worn on a user and measure biological information. In short, the information processing apparatus 10 may be a wearable apparatus. For example, the information processing apparatus 10 may be an apparatus worn on a user's head, a hearable apparatus worn on a user's ear, an apparatus worn on a user's arm, hand, wrist, or finger (such as a watch-type apparatus), an apparatus hung on a user's neck, or an apparatus worn on a user's body or leg.

Similarly, the terminal apparatus 12 may include one or more biological information measurement apparatuses 14. The entire terminal apparatus 12 including one or more biological information measurement apparatuses 14 may be worn on a user and measure biological information. In short, the terminal apparatus 12 may be a wearable apparatus. For example, the terminal apparatus 12 may be an apparatus worn on a user's head, a hearable apparatus worn on a user's ear, an apparatus worn on a user's arm, hand, wrist, or finger (such as a watch-type apparatus), an apparatus hung on a user's neck, or an apparatus worn on a user's body or leg.

In the present exemplary embodiment, as an example, it is assumed that the information processing apparatus 10 and the biological information measurement apparatus 14 are separate apparatuses, and that the terminal apparatus 12 and the biological information measurement apparatus 14 are also separate apparatuses. For example, the information processing apparatus 10 is a smart speaker, the terminal apparatus 12 is a portable terminal apparatus such as a smartphone or a tablet PC, and the biological information measurement apparatus 14 is a wearable apparatus worn by a user. Needless to say, these configurations are only examples, and other configurations may be adopted.

In the present exemplary embodiment, in the case where a requester who requests implementation of an activity requests a requestee who is requested to implement the activity to implement the activity, biological information of the requestee who has received the request is measured by the biological information measurement apparatus 14. The information processing apparatus 10 estimates, on the basis of the measured biological information, whether the requestee is able to accept implementation of the activity. The activity includes, for example, a task that involves physical movement, a task that uses brain, and so forth. The category of the concept of activity includes working and playing. Some kind of task involving physical movement and some kind of task using brain may be included in the category of the concept of activity.

Hereinafter, the configuration of the information processing apparatus 10 will be described in detail with reference to FIG. 2. FIG. 2 illustrates the configuration of hardware of the information processing apparatus 10.

The information processing apparatus 10 includes, for example, a communication device 16, a user interface (UI) 18, a storage device 20, and a processor 22. The information processing apparatus 10 may include configurations other than these configurations.

The communication device 16 is a communication interface, and has the function of transmitting data to another apparatus and the function of receiving data transmitted from another apparatus. The communication device 16 may have a wireless communication function or a wired communication function. The communication device 16 may communicate with another apparatus using, for example, short-range wireless communication, or may communicate with another apparatus via a communication channel such as a LAN or the Internet.

The UI 18 is a user interface, and includes a display device and an operation device. The display device is, for example, a liquid crystal display or an electroluminescence (EL) display. The operation device includes, for example, a keyboard, input keys, and an operation panel. The UI 18 may be a UI such as a touchscreen that serves as both a display device and an operation device. In addition, a microphone may be included in the UI 18, and/or a speaker that outputs sound may be included in the UI 18.

The storage device 20 is a device constituting one or more storage areas storing various types of data. The storage device 20 includes, for example, a hard disk drive, various types of memory (such as random-access memory (RAM), dynamic RAM (DRAM), and read-only memory (ROM)), other storage devices (such as optical disks), or a combination thereof. One or more storage devices 20 are included in the information processing apparatus 10.

The storage device 20 stores biological response management information, history management information, and schedule management information.

Biological response management information is information for inferring a user's biological response on the basis of biological information measured from the user. For example, predetermined reference biological information, and biological response information indicating a biological response that a user who has generated the reference biological information is inferred to show are linked in advance with each other and registered in the biological response management information. For each user, reference biological information and biological response information may be linked with each other and registered in the biological response management information.

A biological response is, for example, a response that reflects a user's feeling. For example, a feeling of having fun, a feeling of being relaxed, a feeling of being excited, a feeling of being motivated, and a feeling of having no stress are positive biological responses, that is, favorable biological responses. A feeling of being bored, a feeling of being frustrated, a feeling of wanting to flee, a feeling of being not good at something, a feeling of unmotivated, and a feeling of having stress are negative biological responses, that is, biological responses that represent rejection.

Reference biological information is defined by, for example, statistical processing, and is biological information that is assumed to be generally generated by a user who has a biological response linked with the reference biological information.

History management information is information for managing a biological response of a user who is a requestee who has received a request to implement an activity. For example, for each user, user identification information for identifying a user who is a requestee who has received a request to implement an activity, time and date information indicating the date and time at which the user received the request to implement the activity, activity information regarding the activity, and biological response information indicating a biological response of the user who has received the request to implement the activity are linked with one another and registered in the history management information.

User identification information is, for example, a user's name, user ID, or user account.

Activity information is information indicating what kind of activity the activity is, and includes information indicating the details thereof. In addition, activity information may include information indicating the place where the activity is implemented, information indicating the date and time at which the activity is implemented, information for identifying another or other co-implementers who cooperatively implement the activity, and information for identifying a requester who has made the request for the activity.

For example, whenever a user receives a request to implement an activity, the user's biological information is measured; on the basis of the measured biological information, a biological response is inferred; and biological response information indicating the inferred biological response is registered in the history management information.

Schedule management information is information for managing the schedule of an activity assigned to a user who is a requestee, the schedule of an activity that has not been assigned to anybody, and an already-implemented activity. For example, activity information regarding an upcoming activity and user identification information for identifying a user who will implement the activity are linked with each other and registered in the schedule management information. A user who will implement an activity is a user to which implementation of the activity is assigned. In the case where an activity is implemented, activity information regarding the already-implemented activity, user identification information for identifying a user who implemented the activity, and biological response information indicating a biological response of the user when implementing the activity may be linked with one another and registered in the schedule management information. If a user's biological information when implementing an activity is not measured, biological response information indicating the user's biological response when implementing the activity is not registered in the schedule management information. In addition, information regarding an activity being implemented may be registered in the schedule management information. Information indicating the progress of each activity may be linked with the activity and registered in the schedule management information.

Note that at least one of the biological response management information, the history management information, and the schedule management information may not be stored in the storage device 20, and may be stored in another apparatus (such as a server).

The processor 22 is configured to obtain biological information of a requestee who has received a request to implement an activity, which is generated from the requestee on receipt of the request to implement the activity, and, on the basis of the biological information, estimate whether the requestee is able to accept implementation of the activity. The processor 22 may obtain biological information from the biological information measurement apparatus 14 or obtain biological information from the terminal apparatus 12 which has obtained the biological information from the biological information measurement apparatus 14. The processor 22 may estimate whether the requestee is able to accept implementation of the activity on the basis of one type of biological information, or may estimate whether the requestee is able to accept implementation of the activity on the basis of a plurality of types of biological information.

The processor 22 may manage a history regarding a requestee's biological response on receipt of a request to implement an activity. For example, the processor 22 may register biological response information indicating a requestee's biological response on receipt of a request to implement an activity in the history management information.

The processor 22 may assign implementation of an activity to a user. For example, the processor 22 may link activity information indicating the details of an upcoming activity and user identification information for identifying a user who will implement the activity and register these linked items of information in the schedule management information.

In addition, the processor 22 is configured to control the operation of each unit of the information processing apparatus 10. The processor 22 may include memory.

For example, a brain machine interface may be constituted by the biological information measurement apparatus 14, which measures brain activity, and the information processing apparatus 10. The system of a brain machine interface may be invasive or noninvasive. In this case, the processor 22 infers a user's biological response on the basis of the user's brain activity (such as brain waves). To infer a biological response, the processor 22 may extract a characteristic component from brain waves and infer a biological response on the basis of the extracted component. To extract a characteristic component from brain waves, a fast-Fourier transform (FFT), a wavelet transform (WT), a time-frequency distribution (TFD), an eigenvector method (EM), and an auto-regressive model (ARM) may be used. In addition, as a method of linking brain waves and a biological response using a feature vector obtained by feature extraction, for example, an independent component analysis (ICA), K-means clustering, a support vector machine (SVM), and a convolutional neural network (CNN or ConvNet) may be used.

The terminal apparatus 12 includes, for example, a communication device, a UI, a storage device, and a processor. For example, the processor transmits data to another apparatus using the communication device, receives data from another apparatus using the communication device, displays an image on a display of the UI, receives data input via the UI, and stores data in the storage device.

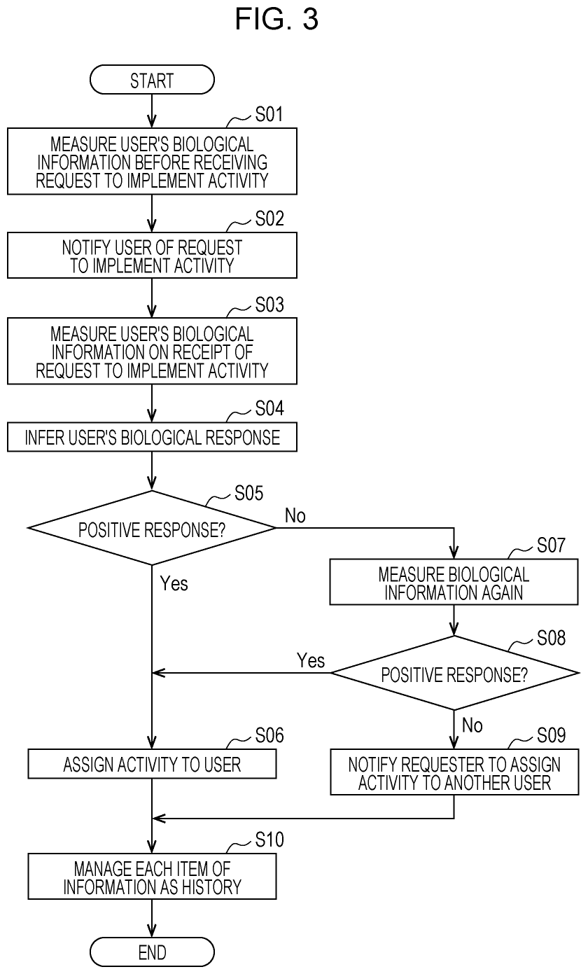

Hereinafter, a process performed by the information processing apparatus 10 will be described in detail with reference to FIG. 3. FIG. 3 is a flowchart illustrating an exemplary process performed by the information processing apparatus 10.

Here, as an example, it is assumed that a user X1, who is a requester, requests a user A1, who is a requestee, to implement an activity. It is also assumed that the terminal apparatus 12A is used by the user A1, who is the requestee; the terminal apparatus 12C is used by the user X1, who is the requester; and the biological information measurement apparatus 14A measures biological information of the user A1.

At first, biological information of the user A1, who is the requestee, before receiving the request to implement the activity, is measured by the biological information measurement apparatus 14A (S01). Note that this measurement is for grasping a biological response of the user A1 before receiving the request to implement the activity, and this measurement need not be performed.

Next, the user A1 is notified of the request to implement the activity (S02).

Here, a method of notifying a user who is a requestee of a request to implement an activity will be described.

For example, the user X1, who is the requester, requests the user A1, who is the requestee, to implement the activity using the terminal apparatus 12C of the user X1.

When the user X1 specifies, using the terminal apparatus 12C of the user X1, the details of the activity being requested and the user A1, who is the requestee, and gives a command to send the request, the terminal apparatus 12C transmits information indicating the request to implement the activity to the information processing apparatus 10. Information indicating the request to implement the activity includes, for example, user identification information of the user X1, who is the requester, activity information regarding the activity being requested, and user identification information of the user A1, who is the requestee. Activity information included in information indicating the request to implement the activity may include only information indicating the details of the activity, or may include, besides information indicating the details of the activity, information indicating the scheduled date and time at which the activity will be implemented, information for identifying another or other co-implementers who are scheduled to cooperatively implement the activity, and information for identifying the requester who has made the request to implement the activity. For example, the user X1 may specify information included in information indicating the request to implement the activity.

On receipt of the information indicating the request to implement the activity from the terminal apparatus 12C, the processor 22 of the information processing apparatus 10 notifies the user A1 of the request to implement the activity. By referring to the information indicating the request to implement the activity, the processor 22 recognizes the user X1, who is the requester, the user A1, who is the requestee, and the details of the activity being requested. As will be described later, the processor 22 registers the information indicating the request to implement the activity as history information in the history management information.

For example, the processor 22 transmits the information indicating the request to implement the activity to the terminal apparatus 12A of the user A1. Address information of the terminal apparatus 12A may be stored in advance in the storage device 20 of the information processing apparatus 10, or may be specified by the user X1 at the time of the request and included in the information indicating the request to implement the activity. The processor of the terminal apparatus 12A which has received the information indicating the request to implement the activity displays the information on the display of the UI of the terminal apparatus 12A. Accordingly, the user A1 is notified of the request to implement the activity. For example, the information indicating the details of the activity, which is included in the activity information, is displayed to allow the user A1 to recognize the details of the activity being requested. In addition, in the case where the activity information includes information other than the information indicating the details of the activity (such as information indicating the date and time at which the activity is implemented), information other than the information indicating the details of the activity may also be displayed. Accordingly, the user A1 may recognize the date and time and the place where the activity being requested is implemented.

As another example, the processor 22 may notify the user A1 of the request to implement the activity by outputting sound from a speaker, or may notify the user A1 of the request to implement the activity by displaying an image on the display of the UI 18.

The user X1 may request the user A1 to implement the activity without using the terminal apparatus 12. For example, when the user X1 notifies the user A1 of the request to implement the activity using sound, the sound is measured by a speaker, and the processor 22 receives audio data indicating the request to implement the activity. By analyzing the audio data, the processor 22 recognizes the user X1, who is the requester, the user A1, who is the requestee, and the details of the activity being requested. For example, it is conceivable that the user X1 orally notifies the user A1 of the details of the activity being requested and the date and time at which the activity is implemented. By analyzing image data generated by capturing images of the user X1 and the user A1 with a camera, the processor 22 may recognize the user X1, who is the requester, and the user A1, who is the requestee.

Next, biological information of the user A1 on receipt of the request to implement the activity is measured by the biological information measurement apparatus 14A (S03). The measured biological information may be transmitted from, for example, the biological information measurement apparatus 14A to the information processing apparatus 10, or may be transmitted from the biological information measurement apparatus 14A to the terminal apparatus 12A of the user A1, and then transmitted from the terminal apparatus 12A to the information processing apparatus 10.

For example, in the case where biological information of the user A1 is continuously measured by the biological information measurement apparatus 14A before the user A1 receives a request for an activity, the processor 22 receives from the biological information measurement apparatus 14A biological information measured from the user A1 within a predetermined period from receipt of the request.

As another example, in the case where biological information of the user A1 is continuously measured by the biological information measurement apparatus 14A before the user A receives a request for an activity and is transmitted to the information processing apparatus 10, the processor 22 may infer, in step S04 described later, a biological response of the user A1 on the basis of biological information measured from the user A1 within a predetermined period from receipt of the request.

As another example, in the case where biological information of the user A1 is not measured by the biological information measurement apparatus 14A before the user A1 receives a request for an activity, the processor 22 which has received information indicating the request to implement the activity may give a command to the biological information measurement apparatus 14A to measure biological information. As another example, the processor of the terminal apparatus 12A which has received information indicating the request to implement the activity may give a command to the biological information measurement apparatus 14A to measure biological information. The biological information measurement apparatus 14A, which has received the command, measures biological information of the user A1 and transmits the biological information to the information processing apparatus 10.

Note that the requester or the requestee may give a command to the information processing apparatus 10 to measure the requestee's biological information. The command may be given using the UI 18, or may be give using the requester's or requestee's terminal apparatus 12. The processor 22, which has received the command, gives a command to the biological information measurement apparatus 14, which measures the requestee's biological information, to measure biological information. The biological information measurement apparatus 14, which has received the command, measures the requestee's biological information and transmits the biological information to the information processing apparatus 10 via or not via the terminal apparatus 12.

On receipt of the biological information measured from the user A1, the processor 22 of the information processing apparatus 10 infers a biological response of the user A1 on the basis of the measured biological information (S04).

For example, the processor 22 compares the biological information measured from the user A1 and each item of reference biological information registered in the above-mentioned biological response management information, and searches for reference biological information whose difference from the biological information measured from the user A1 is included in an allowable range. The processor 22 identifies a biological response linked with the reference biological information found by the search. The allowable range is determined in advance. The allowable range may be changed by the administrator or the like.

In the case where a plurality of items of reference biological information whose differences from the biological information measured from the user A1 are included in the allowable range are found by the search, the processor 22 identifies reference biological information whose difference from the biological information measured by the user A1 is minimum among the plurality of items of reference biological information. The processor 22 identifies a biological response linked with the identified reference biological information.

Reference biological information may be information indicating a characteristic component of biological information. In this case, the processor 22 may extract a characteristic component from the biological information measured from the user A1, and may search for reference biological information whose difference from the extracted component is included in an allowable range.

For example, in the case where brain waves are used as biopotential information, the processor 22 may extract a characteristic component from the measured brain waves and analyze the component to infer a biological response represented by the brain waves.

In the case where the inferred biological response is a positive biological response (YES in S05), the processor 22 assigns the activity being requested to the user A1 (S06). For example, the processor 22 registers activity information indicating the details of the activity being requested and user identification information of the user A1 who has received the request to implement the activity in the schedule management information by linking the activity information with the user identification information. In addition, the processor 22 may transmit information indicating that the activity being requested has been assigned to the user A1 and information indicating a biological response of the user A1 to the terminal apparatus 12C of the user X1. These items of information may be displayed on the display of the UI of the terminal apparatus 12C.

In the case where the inferred biological response is not a positive biological response (NO in S05), biological information of the user A1 is measured again (S07). The processor 22 infers a biological response of the user A1 on the basis of the again-measured biological information.

In the case where the inferred biological response is a positive biological response (YES in S08), the process proceeds to step S06.

In the case where the inferred biological response is not a positive biological response (NO in S08), the processor 22 sends the user X1, who is the requester, a notification prompting the user X1 to assign the activity being requested to another user (S09). For example, the processor 22 transmits information indicating that assigning the activity to another user is recommended to the terminal apparatus 12C of the user X1. The information indicating that assigning the activity to another user is recommended may be displayed on the display of the UI of the terminal apparatus 12C. Note that address information of the terminal apparatus 12C may be stored in advance in the storage device 20 of the information processing apparatus 10, or may be included in the information indicating the request to implement the activity.

The processor 22 registers each item of information in the history management information (S10). For example, the processor 22 registers, in the history management information, user identification information of the user A1, who is the requestee, user identification information of the user X1, who is the requester, activity information regarding the activity being requested, and biological response information indicating a biological response of the user A1 who has received the request. The activity information to be registered is, for example, the entirety or part of the activity information sent by the user X1 to the user A1.

Note that the processing in step S06 need not be performed. In short, the processor 22 need not assign the activity being requested to the requestee. In this case, the processor 22 may transmit information indicating that a biological response of the user A1 is a positive biological response to the terminal apparatus 12C of the user X1. For example, the information may be displayed on the display of the UI of the terminal apparatus 12C, and the user X1 may assign the activity being requested to the user A1 by himself/herself. Alternatively, the user X1 may refer to the biological response of the user A1 and may not necessarily assign the activity being requested to the user A1. In this manner, the requester may refer to a biological response of the requestee who has received the request for the activity and determine whether to assign the activity to the requestee.

Hereinafter, an information processing system according to the present exemplary embodiment will be described in detail by citing specific examples.

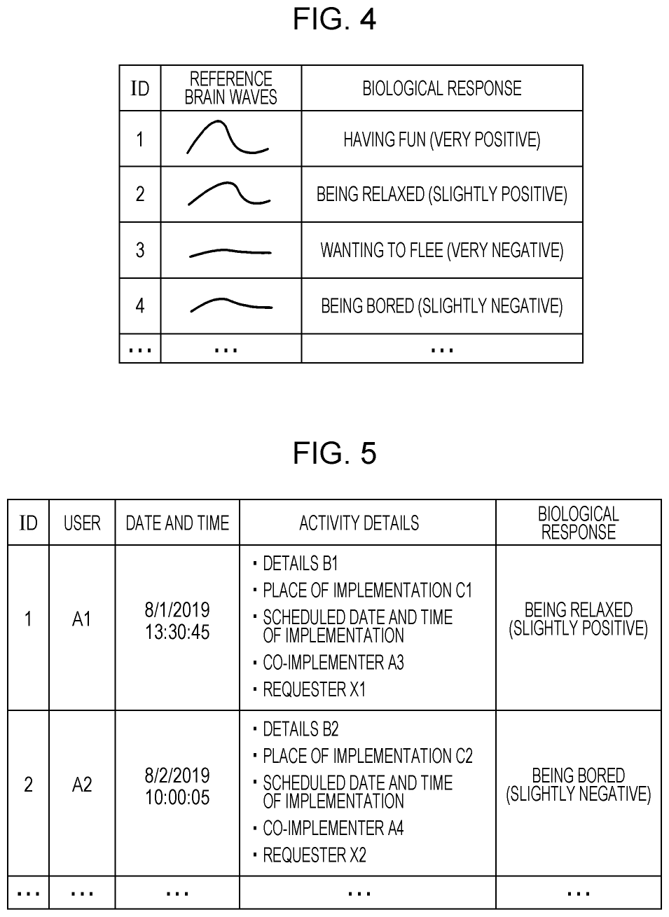

Referring to FIG. 4, the biological response management information will be described in detail. FIG. 4 illustrates an example of a biological response management table, which is an example of the biological response management information. Data of the biological response management table is stored in the storage device 20. Note that the data of the biological response management table may be stored in another apparatus such as a server, instead of being stored in the storage device 20.

In the biological response management table, an identification (ID), reference brain waves, and biological response information indicating a biological response are linked in advance with one another. An ID is information for managing information registered in the biological response management table. Reference brain waves are one example of reference biological information. Reference brain waves are defined by, for example, statistical processing, and are brain waves that are assumed to be generated from a user who has a biological response linked with the reference brain waves. Although brain waves are used as one example of biological information, biological information other than brain waves may be used.

In the example illustrated in FIG. 4, a feeling of having fun, a feeling of being relaxed, a feeling of wanting to flee, and a feeling of being bored are registered as examples of a biological response in the biological response management table. A feeling of having fun and a feeling of being relaxed are examples of a positive biological response. A feeling of wanting to flee and a feeling of being bored are examples of a negative biological response.

In addition, the level of a biological response may be registered in the biological response management table. For example, even a feeling that is classified as a positive response varies in its level of being positive depending on the degree thereof. For example, it is inferred that a feeling of having fun is a very positive biological response, and a feeling of being relaxed is a slightly positive biological response. In short, it is inferred that a feeling of having fun is a feeling with a higher degree of being positive and a higher level of being positive than a feeling of being relaxed.

Similarly, even a feeling that is classified as a negative response varies in its level of being negative depending on the degree thereof. For example, it is inferred that a feeling of wanting to flee is a very negative biological response, and a feeling of being bored is a slightly negative biological response. In short, it is inferred that a feeling of wanting to flee is a feeling with a higher degree of being negative and a higher level of being negative than a feeling of being bored.

The level of being positive and the level of being negative may be represented by numerals or the like.

The above-described inference is only one example, and, depending on how a feeling is defined, classifications and/or levels different from those in the above example may be defined.

To explain this with a specific example, reference brain waves linked with the ID “1” are brain waves that represent a very positive biological response of having fun. Reference brain waves linked with the ID “3” are brain waves that represent a very negative biological response of wanting to flee.

For example, in the case where brain waves whose difference from reference brain waves linked with the ID “1” is included in an allowable range are measured from a user, the processor 22 identifies a biological response of “having fun” linked with the reference brain waves, and infers that this user has a feeling of having fun.

In addition, the processor 22 may calculate the degree of similarity between brain waves measured from a user and reference brain waves and determine whether this degree of similarity is greater than or equal to a threshold. The threshold is a value corresponding to the allowable range. In the case where the degree of similarity between the two types of brain waves is greater than or equal to the threshold, the processor 22 determines that the two types of brain waves are similar and determines that the difference between the brain waves measured from the user and the reference brain waves is included in the allowable range. For example, in the case where brain waves whose degree of similarity with reference brain waves linked with the ID “1” is greater than or equal to the threshold are measured from a user, the processor 22 identifies a biological response of “having fun”.

For each user, reference biological information and biological response information may be linked with each other and registered in the biological response management table. For example, biological information measured from a user is registered as the user's reference biological information in the biological response management table. In addition, biological response information indicating the user's biological response at the time the biological information was measured is linked with the reference biological information and is registered in the biological response management table. For example, a person who performs a registration work asks a user about a feeling that the user had when biological information was measured, and registers information indicating this feeling as biological response information in the biological response management table by linking the biological response information with the biological information which is the reference biological information.

Although a biological response is inferred on the basis of brain waves in the example illustrated in FIG. 4, a biological response may be inferred on the basis of other biological information. For example, the processor 22 may infer a user's biological response on the basis of a plurality of types of biological information. For example, the level of stress that a user feels may be measured by analyzing salivary amylase activity contained in the user's saliva. Alternatively, the level of stress that a user feels may be measured using other stress markers. On the basis of the level of stress that a user feels, the processor 22 may infer whether the user is showing a positive or negative response, or infer the level of being positive or negative.

Hereinafter, referring to FIG. 5, the history management information will be described in detail. FIG. 5 illustrates an example of a history management table, which is an example of the history management information. Data of the history management table is stored in the storage device 20. Note that the data of the history management table may be stored in another apparatus such as a server, instead of being stored in the storage device 20.

In the history management table, an ID, user identification information, date and time information, activity information, and biological response information are linked with one another. An ID is information for managing information registered in the history management table. User identification information is user identification information of a user who is a requestee that has received a request for an activity. Date and time information is information indicating the date and time at which a requestee received a request for an activity. Activity information is information indicating the details of an activity being requested. Biological response information is information indicating a biological response inferred on the basis of biological information measured from a user who is a requestee that has received a request for an activity.

Activity information is information indicating, for example, the details of an activity (such as the details of a job), the place where the activity is implemented, the scheduled date and time at which the activity will be implemented, another or other co-implementers who cooperatively implement the activity, and a requester who has made the request to implement the activity. For example, when making a request for an activity, a requester specifies these items of information using the requester's terminal apparatus 12. As another example, activity information may be determined in advance and registered in the schedule management information.

When a requester gives a notification command for the request, activity information is sent from the requester's terminal apparatus 12 to the information processing apparatus 10, and the processor 22 registers the activity information in the history management table.

For example, in the case where the user X1, who is a requester, requests the user A1 to implement an activity with details B1, the processor 22 registers activity information regarding the activity as activity information of the ID “1” in the history management table.

In addition, the processor 22 transmits the activity information to the requestee's terminal apparatus 12. The display of the UI of the terminal apparatus 12 displays the activity information. Accordingly, the requestee is notified of the request for the activity. The processor 22 infers a biological response on the basis of biological information measured from the requestee, and registers biological response information indicating the biological response in the history management table by linking the biological response information with the above-mentioned activity information.

For example, the activity information of the ID “1” is sent from the information processing apparatus 10 to the terminal apparatus 12A of the user A1, and is displayed on the display of the UI of the terminal apparatus 12A. The processor 22 infers a biological response on the basis of biological information of the user A1, and registers biological response information indicating the biological response as biological response information of the ID “1” in the history management table. In addition, the processor 22 registers date and time information indicating the date and time at which the biological information was measured as date and time information of the ID “1” in the history management table.

What are linked with the ID “1” include user identification information of the user A1, who is the requestee, date and time information indicating the date and time at which the user A1 received the request, activity information indicating the details of the activity for which the user X1 has made a request of the user A1, and biological response information indicating a biological response that the user A1 showed on receipt of the request for the activity.

Note that an activity B1 indicated by activity information linked with the ID “1” is an activity scheduled to be implemented at a place C1 with a co-implementer A3.

The user A1 shows a slightly positive response to this activity. Information indicating this response may be sent from the information processing apparatus 10 to the terminal apparatus 12C of the user X1 and displayed on the terminal apparatus 12C. In addition, the processor 22 may assign the activity indicated by the activity information linked with the ID “1” to the user A1.

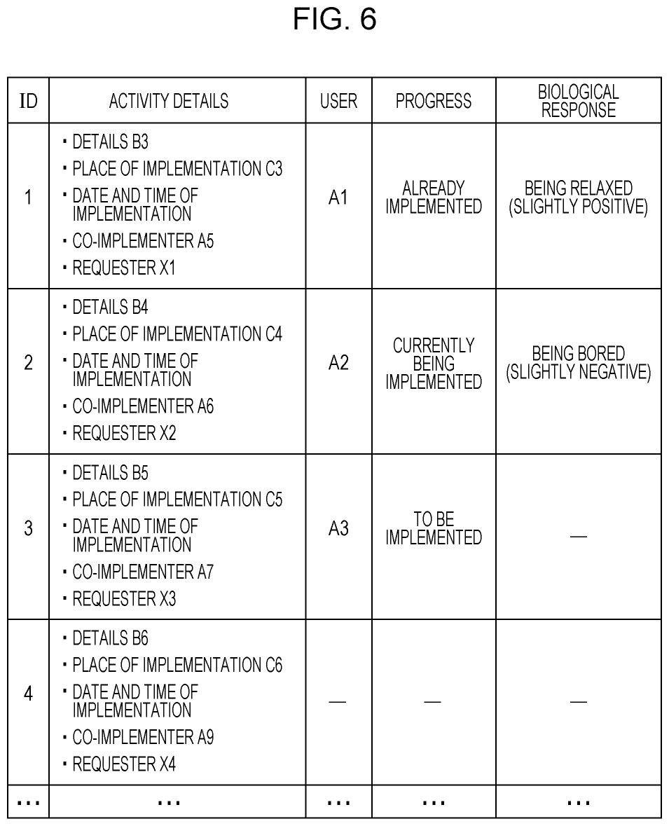

Hereinafter, referring to FIG. 6, the schedule management information will be described in detail. FIG. 6 illustrates an example of a schedule management table, which is an example of the schedule management information. Data of the schedule management table is stored in the storage device 20. Note that the data of the schedule management table may be stored in another apparatus such as a server, instead of being stored in the storage device 20.

What are registered in the schedule management table include the schedule of an activity assigned to a user who is a requestee, the schedule of an activity that has not been assigned to anybody, and an already-implemented activity.

In the schedule management table, for example, an ID, activity information indicating the details of an activity, user identification information of a user who is a requestee, progress information, and biological response information are linked with one another.

Progress information is information indicating the progress of an activity indicated by activity information linked with the progress information. For example, in the case where an activity has been already implemented, the fact that the activity has been already implemented is registered in the schedule management table. In the case where an activity is being implemented, the fact that the activity is being implemented is registered in the schedule management table. In the case where an activity has not been implemented and will be implemented, the fact that the activity will be implemented is registered in the schedule management table.

Biological response information is information indicating a biological response inferred on the basis of biological information measured from a requestee when the requestee is implementing an activity indicated by activity information linked with the biological response information.

For example, an activity linked with the ID “1” is an activity assigned to the user A1 and implemented by the user A1. A biological response of the user A1 when implementing the activity is registered in the schedule management table.

An activity linked with the ID “2” is an activity assigned to the user A2 and currently being implemented by the user A2. A biological response of the user A2 when implementing the activity is registered in the schedule management table.

An activity linked with the ID “3” is an activity assigned to the user A3 and not implemented by the user A3.

An activity linked with the ID “4” is an activity that has not been assigned to anybody.

For example, in the case where an activity linked with the ID “4” is assigned to a new requestee, the requestee's user identification information is linked with the ID “4”.

Note that the schedule may be managed by another apparatus such as a server, instead of being managed by the information processing apparatus 10.

Note that the schedule may be described in data representing a calendar, and the calendar may be used instead of the schedule management table.

Hereinafter, a process according to each example will be described.

### First Example

In a first example, in the case where biological information measured from a requestee (such as the user A1) indicates a response of rejection to implement an activity being requested, the processor 22 assigns the activity to another user other than the requestee. A response of rejection to implement an activity being requested is a biological response that belongs to a negative biological response.

Another user is, for example, a user registered in advance in the information processing apparatus 10 or a user registered in advance in the schedule management table.

In addition, another user may be a user who generated in the past biological information indicating a favorable response to the same activity as the activity being requested or an activity similar to the activity being requested. A favorable response is a biological response that belongs to a positive biological response.

The same activity as the activity being requested is, for example, an activity that has the same details as the activity being requested and that has the same place of implementation. A co-implementer and a requester of the same activity as the activity being requested may be different from those of the activity being requested.

An activity similar to the activity being requested is, for example, an activity that has details similar to the activity being requested. In addition, an activity similar to the activity being requested may be an activity that has the same details as the activity being requested and that has a different place of implementation. For example, for each activity, an activity similar to the activity is determined in advance, and information indicating an activity similar to the activity is stored in the storage device 20.

The processor 22 searches the schedule management table for a user who implemented in the past the same activity as or an activity similar to the activity being requested and who showed a positive biological response while implementing the activity.

The processor 22 assigns the activity being requested to the user found by the search. The processor 22 may notify the user found by the search of information indicating the request to implement the activity, and, if the user approves the assigning of the activity, may assign the activity to the user. For example, the processor 22 transmits the information indicating the request to implement the activity to the terminal apparatus 12 of the user found by the search. The display of the UI of the terminal apparatus 12 displays activity information included in the information indicating the request to implement the activity. In the case where the user found by the search approves, using the terminal apparatus 12, the assigning of the activity, information indicating the approval is sent from the terminal apparatus 12 to the information processing apparatus 10. On receipt of the information indicating the approval, the processor 22 assigns the activity being requested to the user found by the search.

In the case where a requester (such as the user X1) approves the assigning of an activity being requested to a user found by the search, the processor 22 may assign the activity being requested to the user found by the search. For example, the processor 22 may transmit, to the terminal apparatus 12 of a requester (such as the user X1), information indicating that a requestee showed a negative biological response, and information indicating that the activity being requested will be assigned to a user found by the search. The display of the UI of the requester's terminal apparatus 12 displays these items of information. In the case where the requester approves, using the requester's terminal apparatus 12, the assigning of the activity to a user found by the search, information indicating the approval is sent from the requester's terminal apparatus 12 to the information processing apparatus 10. On receipt of the information indicating the approval, the processor 22 assigns the activity being requested to a user found by the search.

In the case where a plurality of users are found by the search, the processor 22 may assign the activity being requested to a user who showed the most positive biological response among the plurality of users, or a user whose level of being positive is the lowest among the plurality of users. In the case where a plurality of user who showed a positive biological response are found by the search, the processor 22 may assign the activity being requested to a user who is most experienced in the activity among the plurality of users, or a user who is least experienced in the activity among the plurality of users. In addition, the requester may be notified of information indicating a plurality of users found by the search, and may specify a user to which the activity being requested is assigned among the plurality of users.

### Second Example

In a second example, in the case where a plurality of requestees received a request to implement an activity, the processor 22 assigns the activity to a requestee who generated biological information indicating a more favorable response to implementation of the activity among the plurality of requestees.

For example, in the case where the number of users implementing the activity is limited, the processor 22 assigns the activity to one or more users who show a more positive biological response than the other users. For example, the processor 22 assigns the activity sequentially to users in descending order of level of being positive until the number of users reaches the maximum number of users implementing the activity.

This will be described by citing a specific example. For example, it is assumed that the number of users implementing a certain activity is one. In short, it is assumed that one user implements the activity.

For example, it is assumed that the user X1, who is a requester, requests the users A1 and A2, who are requestees, to implement an activity. The user X1 specifies the number of users implementing the activity (one in this case). Information indicating the request to implement the activity includes information indicating the number of users specified by the user X1 (one in this case). The information indicating the request to implement the activity is transmitted from, for example, the terminal apparatus 12C of the user X1 to the information processing apparatus 10, and is transmitted from the information processing apparatus 10 to the terminal apparatus 12A of the user A1 and the terminal apparatus 12B of the user A2. This allows the users A1 and A2 to recognize that the request to implement the activity has been received. In addition, the processor 22 recognizes that the number of users implementing the activity being requested is one.

Biological information of the user A1 is measured by the biological information measurement apparatus 14A, biological information of the user A2 is measured by the biological information measurement apparatus 14B, and these items of biological information are sent to the information processing apparatus 10. Note that one biological information measurement apparatus 14 may measure items of biological information of the users A1 and A2.

On the basis of the biological information measured from the user A1, the processor 22 infers a biological response of the user A1 on receipt of the request to implement the activity; and, on the basis of the biological information measured from the user A2, the processor 22 infers a biological response of the user A2 on receipt of the request to implement the activity.

The processor 22 assigns the activity to, of the users A1 and A2, a user who shows the most positive biological response to implementation of the activity being requested. For example, in the case where a biological response of the user A1 is a very positive biological response and a biological response of the user A2 is a slightly negative biological response, the processor 22 assigns the activity to the user A1.

The user X1, who is the requester, may be notified of items of information indicating biological responses of the users A1 and A2. In the case where the user X1 approves the assigning of the activity to the user A1, the processor 22 may assign the activity to the user A1. Instead of the processor 22 assigning the activity to the user A1, the user X1 who has been notified may assign the activity to the user A1.

In the case where a biological response of the user A1 and a biological response of the user A2 are the same, biological information of the users A1 and A2 may be individually measured again, and a biological response of the users A1 and A2 may be individually inferred. The processor 22 assigns the activity to a user whose level of being positive is higher. Measurement of biological information and inference of a biological response may be repeated until there is a difference between a biological response of the user A1 and a biological response of the user A2.

### Third Example

In a third example, in the case where a requestee received a request to implement a plurality of activities that are to be implemented in overlapping time periods, the processor 22 assigns, to the requestee, among the plurality of activities, an activity that makes the requestee generate biological information indicating the most favorable response on receipt of the implementation request.

Implementation of a plurality of activities that are to be implemented in overlapping time periods may be requested by the same requester or a plurality of requesters. For example, the same requester may request a requestee to implement a plurality of activities that are to be implemented in overlapping time periods. Alternatively, in the case where a plurality of requesters each request a requestee to implement one or more activities, the requested activities may have overlapping implementation time periods.

The fact that activities are to be implemented in overlapping time periods means that the time periods in which these activities are implemented partially overlap, or the time periods in which these activities are implemented exactly match. A time period may be the concept including day, week, month, and year.

For example, activity information included in information indicating a request to implement an activity includes, as date and time information indicating a scheduled date and time at which the activity will be implemented, date and time information indicating a scheduled time period. A scheduled time period may be specified by a requester, or may be registered in advance in the schedule management table. The processor 22 recognizes the scheduled implementation time period of each activity by referring to date and time information included in activity information of each activity being requested.

For example, it is assumed that a requestee received a request to implement an activity W1 and a request to implement an activity W2. In the case where the time period in which the activity W1 is to be implemented and the time period in which the activity W2 is to be implemented partially overlap, if the requestee's biological response to the activity W1 is a positive biological response and the requestee's biological response to the activity W2 is a negative biological response, the processor 22 assigns the activity W1 to the requestee.

The processor 22 may notify the requester of information indicating the requestee's individual biological responses to the activities W1 and W2. In the case where the requester approves the assigning of the activity W1 to the requestee, the processor 22 may assign the activity W1 to the requestee. In the case where the requestee approves the assigning of the activity W1 to the requestee himself/herself, the processor 22 may assign the activity W1 to the requestee.

When an activity has already been assigned to a user, if the user is requested to implement a different activity whose implementation time period overlaps the implementation time period of the already-assigned activity, the processor 22 may update the assigning of the activities. For example, in the case where the user's biological response to the later-requested different activity is a more positive biological response than the user's biological response to the already-assigned activity, the processor 22 updates the assigning of the activities. Specifically, the processor 22 removes the assigning of the already-assigned activity from the user, and assigns the later-requested different activity to the user. In this case, in the case where the requestee's or requester's approval is obtained, the processor 22 may update the assigning of the activities. Note that the activity whose assigning has been removed is assigned to another user.

Note that a plurality of requesters each may request a plurality of requestees to implement one or more activities. In this case, for each requestee, the processor 22 assigns, to the requestee, activities whose implementation time periods do not overlap and to which more positive biological responses are obtained.

### Fourth Example

Information indicating a biological response to an activity, which is registered in the history management table and the schedule management table, may be used as, for example, information for determining a user who implements the same activity as the activity or an activity similar to the activity.

For example, a requester may determine a requestee who is the destination of a request to implement an activity by referring to the history management table, the schedule management table, and information indicating a biological response extracted from each table.

### Fifth Example

In some cases, a requestee's state indicated by biological information measured from the requestee before receiving a request to implement an activity is not included in a predetermined state range. In this case, after waiting for the requestee's state indicated by biological information measured from the requestee to be included in the range, the processor 22 may estimate whether the requestee is able to accept implementation of the activity being requested.

Being within the predetermined state range means, for example, being within the range of a human normal state. For example, the state of a user showing a very positive biological response and the state of a user showing a very negative biological response are not included in the range of a normal state. For example, a state in which the level of being positive is lower than a very positive biological response (such as the state of a user showing a slightly positive biological response) and a state in which the level of being positive is higher than a very negative biological response (such as the state of a user showing a slightly negative biological response) are included in the range of a normal state. In addition, a state in which a pulse or blood pressure is included in a certain range may be within the range of a normal state.

For example, each user's biological information is continuously measured by the biological information measurement apparatus 14 and is transmitted to the information processing apparatus 10. The processor 22 continuously recognizes each user's biological response. On receipt of information indicating a request to implement an activity from a requester, the processor 22 determines, on the basis of a requestee's biological response, whether the requestee's state is included in the range of a normal state. In the case where the requestee's state is not included in the range of a normal state, the processor 22 waits for the requestee's state to be included in the range of a normal state, and then notifies the requestee of the request to implement the activity. For example, after the requestee's state becomes a state within the range of a normal state, the processor 22 notifies the requestee of the request to implement the activity. In the case where the requestee's state is included in the range of a normal state on receipt of information of the request to implement the activity, the processor 22 notifies the requestee of the request to implement the activity.

In addition, in the case where the requestee's state is not included in the range of a normal state, the processor 22 may transmit warning information to the requester's terminal apparatus 12. The warning information may be displayed on, for example, the display of the UI of the terminal apparatus 12. This allows the requester to recognize that the requestee's state is not included in the range of a normal state.

In the case where the requestee's state is not included in the range of a normal state, when the requestee is notified of the request to implement the activity, and biological information of the requestee who has received the request to implement the activity is measured to infer a biological response, due to the influence of the requestee's state before the requestee receives the request to implement the activity, the inferred biological response may not accurately represent the requestee's original biological response on receipt of the request to implement the activity. For example, in the case where the requestee's biological response before receiving the request to implement the activity is a very positive biological response, even if the requestee's original biological response to the activity is a slightly negative biological response, it is conceivable that a biological response to be inferred may be a slightly positive biological response due to the influence of the biological response before receiving the request to implement the activity. When a biological response is inferred after waiting for the requestee's state to be included in the range of a normal state, the requestee's original biological response to the request to implement the activity may be inferred.

### Sixth Example

In a sixth example, the processor 22 may adjust the amount of an activity being requested in accordance with the level of a biological response indicated by biological information measured from a requestee, and assign the activity to the requestee.

For example, in the case where a biological response of a requestee receiving a request to implement an activity belongs to a negative biological response, the processor 22 reduces the amount of the activity and assigns the activity to the requestee. The amount to be reduced is an amount in accordance with the level of a biological response. For example, the processor 22 reduces a greater amount as the level of being negative becomes higher.

In addition, the processor 22 may infer the requestee's biological response on the basis of biological information measured after reducing the amount of the activity. If this biological response belongs to a positive biological response, the processor 22 may assign the amount-reduced activity to the requestee. In short, the processor 22 may reduce the amount of the activity until the requestee's biological response becomes a positive biological response and assign the activity to the requestee.

In addition, the processor 22 may notify the requester of reduction of the amount of the activity. In the case where the requester approves reduction of the amount of the activity, the processor 22 may assign the amount-reduced activity to the requestee.

As another example, the processor 22 may adjust the difficulty of an activity being requested in accordance with the level of a biological response indicated by biological information measured from a requestee, and assign the activity to the requestee. It may be said that the difficulty of an activity is the quality of an activity.

For example, in the case where a biological response of a requestee receiving a request to implement an activity belongs to a negative biological response, the processor 22 reduces the difficulty of the activity and assigns the activity to the requestee. In short, the processor 22 may assign an activity with difficulty according to the level of a biological response to the requestee. The difficulty to be reduced is a degree according to the level of a biological response. For example, the processor 22 assigns an activity with lower difficulty to the requestee as the level of being negative becomes higher. Reduction of difficulty means lowering of difficulty.

In addition, the processor 22 may infer the requestee's biological response on the basis of biological information measured after reducing the difficulty. If this biological response belongs to a positive biological response, the processor 22 may assign the difficulty-reduced activity to the requestee. In short, the processor 22 may reduce the difficulty of the activity until the requestee's biological response becomes a positive biological response and assign the activity to the requestee.

In addition, the processor 22 may notify the requester of reduction of the difficulty. In the case where the requester approves reduction of the difficulty, the processor 22 may assign the difficulty-reduced activity to the requestee.

The processor 22 may adjust the amount or difficulty of or both the amount and difficulty of an activity being requested in accordance with the level of a biological response indicated by biological information measured from a requestee.

The processor 22 may adjust a time period in which an activity being requested is implemented. For example, the processor 22 may infer a biological response on the basis of biological information measured from a requestee while changing a time period in which an activity being requested is implemented. In short, the processor 22 infers a biological response for each time period. For example, the processor 22 changes a time period in which an activity being requested is implemented to a time period in which a positive biological response is obtained. The processor 22 may notify the requester that the time period will be changed. In the case where the requester approves the change of the time period, the processor 22 may assign the activity whose implementation time period has been changed to the requestee.

When the amount of an activity is reduced, the processor 22 or the requester may assign the reduced amount of the activity to another requestee.

### Seventh Example

In a seventh example, a requestee's biological information is measured while the requestee is implementing an activity being requested. In the case where this biological information indicates a response of rejection to implement the activity, the processor 22 notifies a requester who made the request to implement the activity of a warning.

For example, in the example illustrated in FIG. 6, an activity of the ID “2” is being implemented by the user A2; and a biological response of the user A2 is a negative biological response of being bored. In this case, the processor 22 transmits warning information to the terminal apparatus 12 of the user X2, who is a requester. The warning information is, for example, information indicating that the user A2, who is a requestee, is showing a negative biological response. The display of the UI of the terminal apparatus 12 of the user X2 displays the warning information. This allows the user X2 to recognize that the user A2, who is the requestee, has a negative impression on the activity assigned to the user A2. With reference to the warning information, the user X2, who is the requester, may request another user to implement the activity of the ID “2” or may adjust the amount of the activity assigned to the user A2.

In the case where a requestee shows a biological response of rejection to implement an activity while implementing the activity, the processor 22 may reduce the amount of the activity. Using the above-described example, the processor 22 reduces the amount of the activity of the ID “1” and assigns the activity to the user A2. Like the sixth example, the processor 22 may reduce the amount of the activity in accordance with the level of a biological response.

In the case where a requestee shows a biological response of rejection to implement an activity while implementing the activity, the processor 22 may reduce the difficulty of the activity. Using the above-described example, the processor 22 reduces the difficulty of the activity of the ID “1” and assigns the activity to the user A2. Like the sixth example, the processor 22 may reduce the difficulty of the activity in accordance with the level of a biological response.

In the case where a requestee shows a biological response of rejection to implement an activity while implementing the activity, the processor 22 may reduce the amount or difficulty of or both the amount and difficulty of the activity.

In the case where a requestee shows a biological response of rejection to implement an activity while implementing the activity, the processor 22 may assign the activity to another user. The processor 22 may further assign the activity to another user while assigning the activity to the originally requested requestee. In short, the processor 22 may increase the number of users implementing the activity. As another example, the processor 22 may remove the assigning of the activity to the originally requested requestee, and newly assign the activity to another user. As yet another example, the processor 22 may reduce the amount of the activity while assigning the activity to the originally requested requestee, and, if the requestee still shows a biological response of rejection, may assign the activity to another user. The significance of another user is the same as the significance of another user according to the first example.

### Eighth Example

In an eighth example, in the case where the place where an activity is implemented is specified, if a biological response of a requestee who has received a request to implement the activity is different from the requestee's past biological response to the same activity as the activity or an activity similar to the activity, the processor 22 notifies a requester who made the request to implement the activity of the fact that the place where the activity being requested is implemented is a factor that makes the requestee's biological response different. In addition, the processor 22 notifies the requester that the requestee's biological response is different from the past biological response.

The past biological response is, for example, a biological response inferred from the requestee's biological information when the requestee has received in the past a request for the same activity as or an activity similar to the activity being requested, and information indicating the biological response is registered in the history management table. As another example, the past biological response may be a biological response inferred from biological information measured while the requestee was implementing, in the past, the same activity as or an activity similar to the activity being requested.

For example, in the case where the past biological response is a positive biological response and a biological response to the activity being requested is a negative biological response, the processor 22 notifies the requester of the fact that the place where the activity being requested is implemented is a factor that makes the requestee's biological response different. In addition, the processor 22 notifies the requester of the fact that the past biological response is a positive biological response and a biological response to the activity being requested is a negative biological response.

This will be described by citing a specific example. For example, it is assumed that the user X1, who is a requester, requests the user A1, who is a requestee, to implement an activity. It is also assumed that the user A1 has implemented in the past the same activity as or an activity similar to the activity being requested. In this case, in the case where a biological response of the user A1 who has received the request to implement the activity being requested is a negative biological response and the past biological response is a positive biological response, the processor 22 notifies the user X1 of the fact that the place where the activity being requested is implemented is a factor that makes the user A1's biological response different. In addition, the processor 22 notifies the user X1 of the fact that the past biological response of the user A1 is a positive biological response and a biological response of the user A1 to the activity being requested is a negative biological response. The processor 22 may also notify the user X1 of the place where an activity was implemented in the past. Because activity information regarding an activity implemented in the past is registered in the history management table, the processor 22 obtains, from the history management table, information indicating the place where an activity was implemented in the past, and transmits the information to the terminal apparatus 12 of the user X1.

In the case where a biological response of a requestee who has received a request to implement an activity is a biological response of rejection to implement the activity, the processor 22 may give the requester guidance on a candidate for a place where a favorable biological response has been obtained.

For example, it is assumed that the user A1, who received a request to implement an activity, shows a negative biological response, and the place where the activity is implemented is the place C1. It is also assumed that the same activity as or an activity similar to the activity being requested was implemented in the past by the user A1 at a place C2, and the past biological response is a positive biological response. In this case, the processor 22 notifies a requester to change the place where the activity being requested is implemented from the place C1 to the place C2, and to assign the activity again. For example, the processor 22 transmits information indicating the place C2 as a recommended place to the requester's terminal apparatus 12. This allows the requester to recognize a place where the user A1 shows a positive biological response.

### Ninth Example

In a ninth example, an activity being requested is scheduled to be implemented by a plurality of users, and activity information regarding the activity being requested includes user identification information for identifying one or more co-implementers. The activity information is sent to the requestee's terminal apparatus 12, and is displayed on the display of the UI of the requestee's terminal apparatus 12. This allows the requestee to recognize another or other requestees (that is, co-implementers) who cooperatively implement the activity being requested.

In the case where the requestee is able to recognize the co-implementer(s), if the requestee's biological response is a biological response of rejection to implement the activity being requested (such as a negative biological response), the processor 22 notifies a requester of user identification information of the requestee and user identification information of the co-implementer(s) other than the requestee. In addition, the processor 22 also notifies the requester of the fact that the requestee's biological response is a biological response of rejection to implement the activity being requested. This allows the requester to infer that the factor that the requestee's biological response indicates rejection is the co-implementer(s) other than the requestee.

In the case where the requestee's biological response is a positive biological response, the processor 22 may notify the requester of the fact that the requestee's biological response is a positive biological response.

For example, the requester may grasp, on the basis of a notification from the information processing apparatus 10, who is or is not compatible with whom.

### Tenth Example

If, after a period during which an activity being requested is implemented, an event expected to fluctuate a biological response of a requestee who has received a request to implement the activity is scheduled, the processor 22 may send a requester a notification prompting the requester to simultaneously present information regarding the event to the requestee and to request the requestee to implement the activity.

The processor 22 recognizes a period during which the activity is implemented on the basis of information indicating the date and time at which the activity is implemented, which is included in activity information regarding the activity being requested. Next, by referring to the schedule management table, the processor 22 checks whether an event that is scheduled after the period during which the activity is implemented and that is expected to fluctuate a biological response of the requestee who has received a request to implement the activity is scheduled. An event expected to fluctuate the requestee's biological response is, for example, an event expected to make the requestee's biological response a positive biological response or an event expected to make the requestee's biological response a negative biological response. Such events are defined in advance. For example, long vacations, paydays, and bonus paydays are events expected to make the requestee's biological response a positive biological response, and many scheduled activities are events expected to make the requestee's biological response a negative biological response.

The requester receiving the notification gives a command, using the requester's terminal apparatus 12, to include information regarding an event that fluctuates the requestee's biological reaction in information indicating the request to implement the activity. Information indicating the command is sent from the terminal apparatus 12 to the information processing apparatus 10. On receipt of the command, the processor 22 includes information regarding an event that fluctuates the requestee's biological response in information indicating the request to implement the activity, and transmits the information indicating the request to implement the activity to the requester's terminal apparatus 12. The display of the UI of the requester's terminal apparatus 12 displays the information regarding the event, along with the information indicating the request for the activity. A biological response inferred from biological information measured at this time is inferred to be a biological response that is a mixture of a biological response to the activity being requested and a biological response to the event. With reference to this biological response, the requester may determine whether to assign the activity to the requestee.

### Other Embodiments

A requestee's biological response to an activity may change depending on the requester. In the case where a biological response of a requestee who has received a request to implement an activity is different from the requestee's past biological response to the same or similar activity as the activity, the processor 22 may notify the requester of the fact that the requester is a factor that makes the requestee's biological response different. With reference to the notification, the requester may determine whether to request the requestee to implement the activity.

The processor 22 may notify, step by step, the requestee the details of the activity being requested. For example, the processor 22 first transmits information indicating the name of the activity being requested to the requestee's terminal apparatus 12, and displays the information indicating the name of the activity being requested on the display of the UI of the terminal apparatus 12. The processor 22 obtains the requestee's biological information at that time and infers a biological response. In the case where it is unknown whether the biological response is a positive biological response or a negative biological response, the processor 22 next transmits information indicating more details of the activity being requested to the requestee's terminal apparatus 12, and displays the information indicating more details on the display of the UI of the terminal apparatus 12. If a biological response at that time is a positive biological response or a negative biological response, the processor 22 transmits information indicating the biological response to the requester's terminal apparatus 12. If a biological response at that time is a positive biological response, the processor 22 may assign the activity to the requestee.

The information processing system according to the present exemplary embodiment may be used for, for example, labor management of employees. For example, the level of stress on an employee when a job is assigned or the level of stress on an employee while doing an assigned job may be measured, and the measurement result may be sent as a feedback for labor management. In addition, the level of stress on an employee after finishing an assigned job may be measured, and the measurement result may be sent as a feedback for labor management. For example, the schedule of a job may be adjusted at any time on the basis of the measurement result.

In addition, the information processing system according to the present exemplary embodiment may be used for managing a user's own actions. For example, in response to a user's operation, information indicating the details of an activity that the user intends to execute is displayed on the display of the UI 18, and biological information of the user seeing the displayed information is measured by the biological information measurement apparatus 14. On the basis of the measured biological information, the user's biological response is inferred, and information indicating the inferred biological response is displayed on the display of the UI 18. This allows the user to recognize the user's own biological response to an action that the user intends to execute. For example, the information processing system according to the present exemplary embodiment may be used to allow employees to understand their own stress levels. For example, before an employee performs a task, the employee may check his/her own biological response to the task.

In the embodiment above, the term “processor” refers to hardware in a broad sense. Examples of the processor includes general processors (e.g., CPU: Central Processing Unit), dedicated processors (e.g., GPU: Graphics Processing Unit, ASIC: Application Integrated Circuit, FPGA: Field Programmable Gate Array, and programmable logic device). In the embodiment above, the term “processor” is broad enough to encompass one processor or plural processors in collaboration which are located physically apart from each other but may work cooperatively. The order of operations of the processor is not limited to one described in the embodiment above, and may be changed.

The foregoing description of the exemplary embodiment of the present disclosure has been provided for the purposes of illustration and description. It is not intended to be exhaustive or to limit the disclosure to the precise forms disclosed. Obviously, many modifications and variations will be apparent to practitioners skilled in the art. The embodiment was chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. An information processing apparatus comprising:
a processor configured to:
obtain biological information of a person who has received a request to implement an activity, the biological information being generated from the person on receipt of the request to implement the activity; and
estimate, on a basis of the biological information, whether the person is able to accept implementation of the activity.

2. The information processing apparatus according to claim 1, wherein, in a case where the biological information indicates a response of rejection to implement the activity, the processor further assigns the activity to an other person other than the person.

3. The information processing apparatus according to claim 2, wherein the other person is a person who has generated in past biological information indicating a favorable response to a same activity as or an activity similar to the activity.

4. The information processing apparatus according to claim 1, wherein, in a case where a plurality of persons receive the request to implement the activity, the processor further assigns the activity to, among the plurality of persons, a person who has generated biological information indicating a most favorable response to implementation of the activity.

5. The information processing apparatus according to claim 1, wherein, in a case where the person has received a request to implement a plurality of activities that are to be implemented in overlapping time periods, the processor further assigns, to the person, among the plurality of activities, an activity that makes the person generate biological information indicating a most favorable response on receipt of the request for implementation.

6. The information processing apparatus according to claim 1, wherein:
the processor further stores, in a storage device, response information indicating the person's response to the activity, which is grasped from the biological information, and
the response information is used as information for determining a user who implements a same activity as or an activity similar to the activity.

7. The information processing apparatus according to claim 1, wherein the biological information is at least one of information indicating a brain activity, electromyogram information, information regarding saliva, information indicating a pulse wave, information indicating a blood pressure, and information indicating a pulse.

8. The information processing apparatus according to claim 2, wherein the biological information is at least one of information indicating a brain activity, electromyogram information, information regarding saliva, information indicating a pulse wave, information indicating a blood pressure, and information indicating a pulse.

9. The information processing apparatus according to claim 3, wherein the biological information is at least one of information indicating a brain activity, electromyogram information, information regarding saliva, information indicating a pulse wave, information indicating a blood pressure, and information indicating a pulse.

10. The information processing apparatus according to claim 1, wherein, in a case where the person's state indicated by biological information generated from the person before receiving the request to implement the activity is not included in a predetermined state range, after waiting for the person's state indicated by the biological information generated from the person to be included in the range, the processor estimates, on a basis of the biological information, whether the person is able to accept implementation of the activity.

11. The information processing apparatus according to claim 1, wherein the processor further adjusts an amount or difficulty of the activity being requested in accordance with a level of a response indicated by the biological information, and assigns the activity to the person.

12. The information processing apparatus according to claim 1, wherein, in a case where biological information generated from the person while implementing the activity indicates a response of rejection to implement the activity, the processor further notifies a requester who has made the request to implement the activity of a warning.

13. The information processing apparatus according to claim 1, wherein, in a case where biological information generated from the person while implementing the activity indicates a response of rejection to implement the activity, the processor further reduces an amount or difficulty of the activity and assigns the activity to the person.

14. The information processing apparatus according to claim 1, wherein, in a case where biological information generated from the person while implementing the activity indicates a response of rejection to implement the activity, the processor further assigns implementation of the activity to an other person.

15. The information processing apparatus according to claim 1, wherein, in a case where a place where the activity is implemented is determined, if a response to the activity, which is indicated by the biological information, is different from the person's past response to a same activity as or an activity similar to the activity, the processor further notifies a requester who has made the request to implement the activity of a fact that the place is a factor that makes the person's response different.

16. The information processing apparatus according to claim 1, wherein, in a case where a place where the activity is implemented is determined, if the biological information indicates a response of rejection to implement the activity, the processor gives guidance on a candidate for a place where biological information indicating a favorable response has been generated from the person.

17. The information processing apparatus according to claim 1, wherein, in a case where information regarding an other person who is scheduled to implement the activity in cooperation with the person is presented to the person, if the biological information indicates a response of rejection to implement the activity, the processor further notifies a requester who has made the request to implement the activity of information regarding the other person who is scheduled to implement the activity in cooperation with the person.

18. The information processing apparatus according to claim 1, wherein, in a case where an event expected to fluctuate the person's response is scheduled after a period during which the activity is implemented, the processor further notifies a requester who makes the request to implement the activity to present information regarding the event to the person and to request the person to implement the activity.

19. The information processing apparatus according to claim 2, wherein, in a case where an event expected to fluctuate the person's response is scheduled after a period during which the activity is implemented, the processor further notifies a requester who makes the request to implement the activity to present information regarding the event to the person and to request the person to implement the activity.

20. A non-transitory computer readable medium storing a program causing a computer to execute a process, the process comprising:
obtaining biological information of a person who has received a request to implement an activity, the biological information being generated from the person on receipt of the request to implement the activity; and
estimating, on a basis of the biological information, whether the person is able to accept implementation of the activity.
